(19) United States
(12) Patent Application Publication
(10) Pub. No.: US 2022/0145287 A1
(43) Pub. Date: May 12, 2022

(54) METHODS AND COMPOSITIONS FOR NEXT GENERATION SEQUENCING (NGS) LIBRARY PREPARATION

(71) Applicant: CO-DIAGNOSTICS, INC., Salt Lake City, UT (US)

(72) Inventor: Jesse L. MONTGOMERY, South Centerville, UT (US)

(21) Appl. No.: 17/605,694

(22) PCT Filed: Apr. 24, 2020
(86) PCT No.: PCT/US2020/029727

§ 371 Date: Oct. 22, 2021

Related U.S. Application Data
(60) Provisional application No. 62/838,036, filed on Apr. 24, 2019.

(51) Int. Cl. C12N 15/10; C12Q 1/6876; C12Q 1/6855; C12Q 1/6806

(52) U.S. Cl. CPC C12N 15/1065; C12Q 1/6806; C12Q 1/6855; C12Q 1/6876

(57) ABSTRACT

Disclosed herein are primer/adapter sequences, wherein each of the primer/adapter sequences comprise a region that hybridizes with a target nucleic acid sequence, as well as an adapter sequence that does not hybridize with the target nucleic acid sequence. Also disclosed are methods of using these primer/adapter sequences to amplify and sequence target nucleic acid sequences in a sample.

### CROSS REFERENCE TO RELATED APPLICATIONS

This application claims priority to U.S. Patent Application No. 62/838,036, filed on Apr. 24, 2019, which is incorporated by reference herein.

### BACKGROUND

Innovations in sequencing technologies over the past decade have been critical driving forces accelerating the ongoing revolution in medicine and the life sciences and opening up new research and business opportunities with boundless potential. The growth of sequencing-based research and business opportunities is highly dependent upon the technological strength of a given sequencing platform.

Nucleic acids are prepared for next generation sequencing (NGS) by adding adapters at the ends of the target sequences. These adapters are specific nucleic acid sequences that allow attachment of targets to the instrument sequencing substrate. Targets prepared with adapter sequences necessary for sequencing are referred to as a library. Adapter sequences differ across instrument manufacturers.

The method for incorporating adapters into target nucleic acids varies depending on the type of NGS performed. For methods intended for NGS that rely on polymerase chain reaction (PCR) to generate nucleic acid targets, (often referred to as targeted amplicon sequencing) PCR is used to sequence selected regions of a genome. This contrasts with shotgun sequencing methods that are intended to sequence all nucleic acid in a sample.

Traditional methods for targeted amplicon sequencing include multiple steps. First, the amplicon is generated. PCR is used to amplify selected targets. Primers for PCR may include partial sequencing adapter sequences. Amplicon preparation follows. This may include cleaning and/or preparation with enzymes. Next is adapter incorporation, which may be done via PCR or incubating with ligase or other enzymes. Last is library cleaning. Unwanted products are removed using one or more methods such as magnetic beads or gel electrophoresis. Of course, following this, sequencing can occur. This process generally takes 7-8 hours with 2-4 hours of hands-on time.

What is needed in the art is a method for targeted amplicon sequencing library preparation that reduces total preparation and user hands-on time.

### SUMMARY

Disclosed herein is a method of preparing a target nucleic acid sequence for targeted amplicon sequencing comprising: a) providing at least one target nucleic acid sequence in a sample; b) exposing the target nucleic acid sequence to at least one pair of primer/adapter sequences, wherein each of the primer/adapter sequences comprise a region that hybridizes with the target nucleic acid sequence, as well as an adapter sequence that does not hybridize with the target nucleic acid sequence; c) amplifying the target nucleic acid in the presence of the primer/adapter sequence pair, thereby incorporating the adapter sequence into copies of the target nucleic acid sequence, creating a target nucleic acid/adapter sequence; d) purifying copies of the target nucleic acid/adapter sequence; and e) exposing the purified target nucleic acid/adapter sequence of step d) to reagents necessary for sequencing.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows an overview of the primer/adapter sequence strategy. The entire adapter sequence (A) is included at the 5′ end of target specific primers (B). The adapters are incorporated into the ends of the target sequences (C) with PCR. Samples are then purified with beads or other methods to remove unincorporated primers and off-target amplification products.

FIG. 2 shows library fragment analysis for Ion Torrent library using primer/adapter sequences. Purity and distribution of library fragments were analyzed with a Fragment Analyzer. Library is shown to be sufficiently pure for sequencing after 1 bead washing.

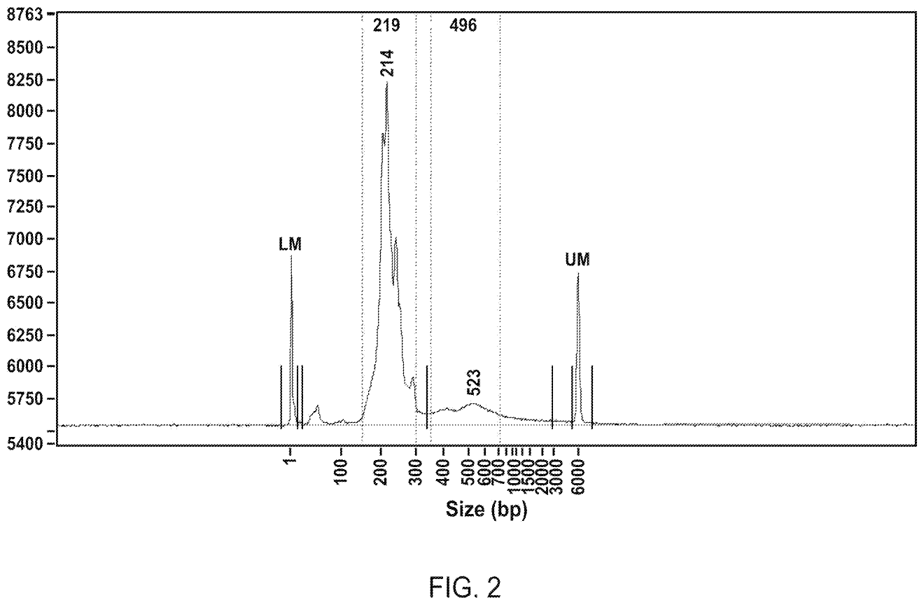

FIG. 3A-B shows target coverage for the Ion Torrent platform using the primer/adapter sequences. Coverage, or the total number of sequences, was calculated at the SNP of interest for each target. This is displayed on a linear scale (A) and a log scale (B) for the y-axis. A large majority of the targets had uniform coverage within two orders of magnitude.

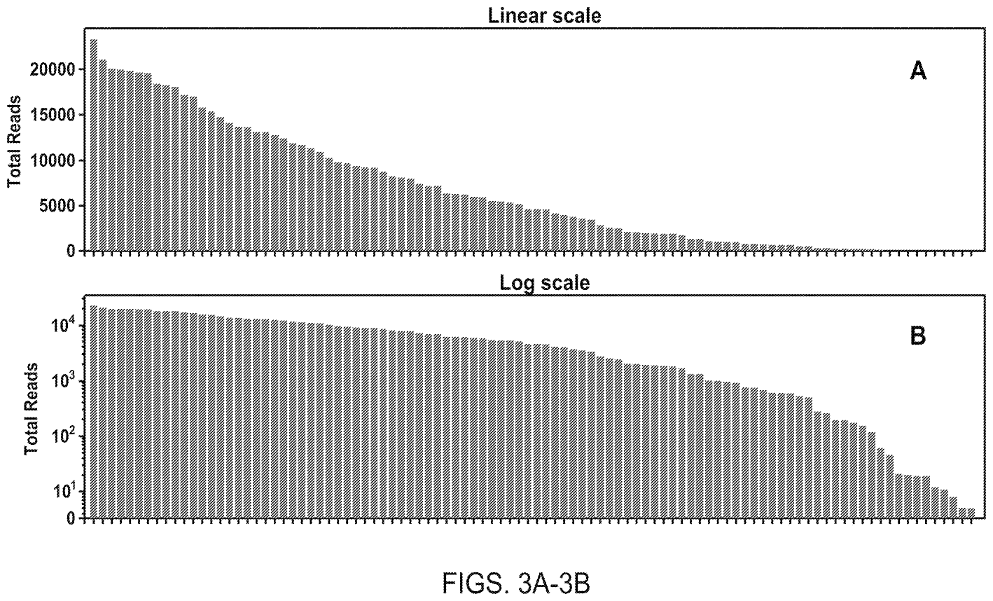

FIG. 4 shows library fragment analysis for Illumina library using primer/adapter sequences. Purity and distribution of library fragments were analyzed with a Fragment Analyzer. Library is shown after two washes. This library was used for sequencing, however one bead washing can be sufficient.

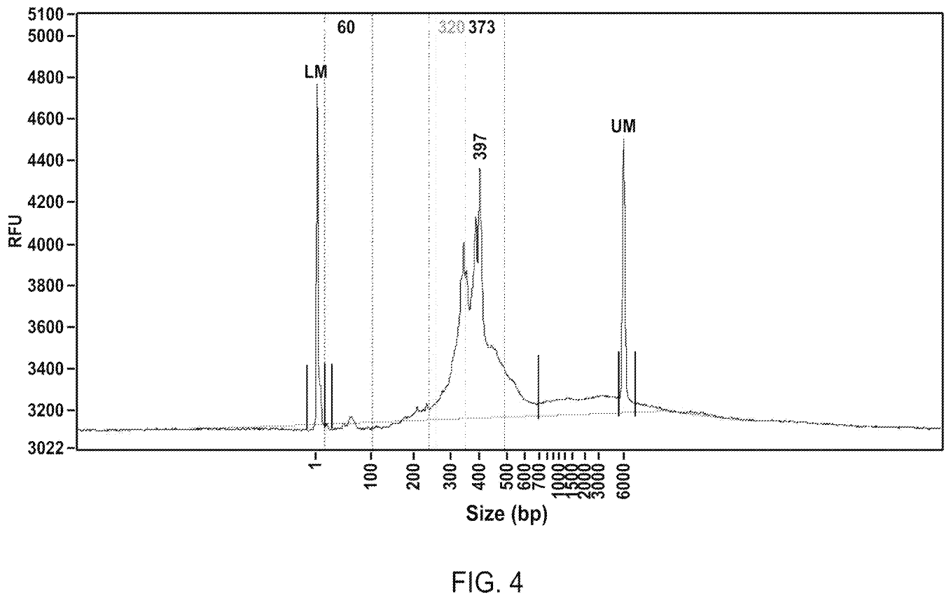

FIG. 5 shows library fragment analysis for Illumina library using primer/adapter sequences. Library is shown after one wash. A short product impurity centered at 61 bases comprises approximately 7% of the total sample.

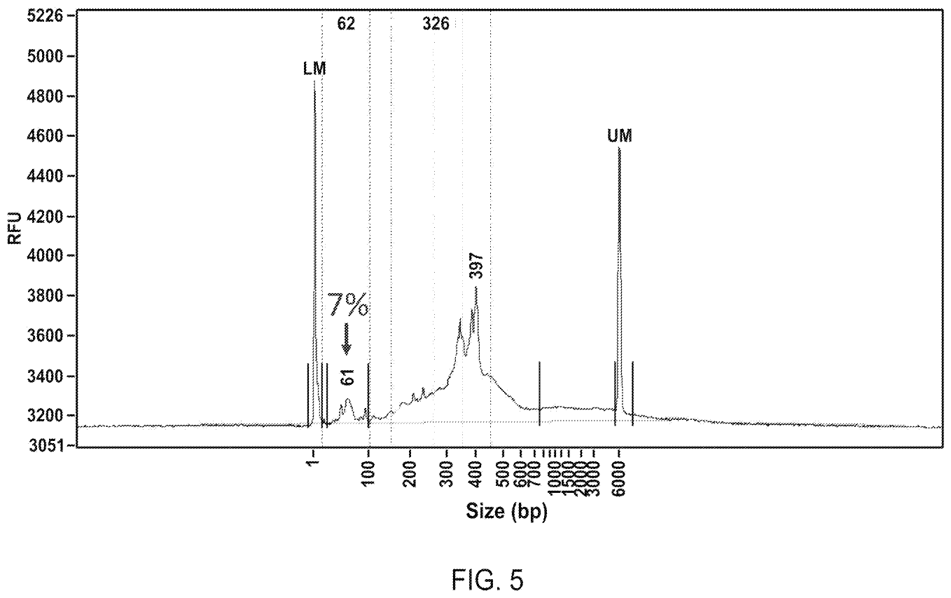

FIG. 6 shows per base read quality for Illumina library using primer/adapter sequences. High quality sequencing is observed for all bases throughout the sequencing fragments. Read 1 is shown.

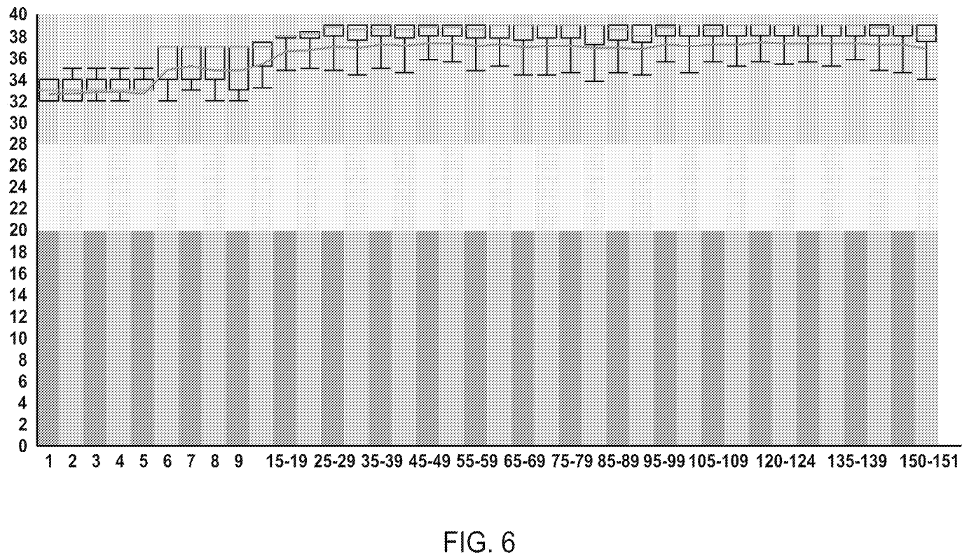

FIG. 7A-B shows target coverage for the Illumina platform using primer/adapter sequences. Coverage, or the total number of sequences, was calculated at the SNP of interest for each target. This is displayed on a linear scale (A) and a log scale (B) for the y-axis. 30 out of 50 targets had uniform coverage within two orders of magnitude.

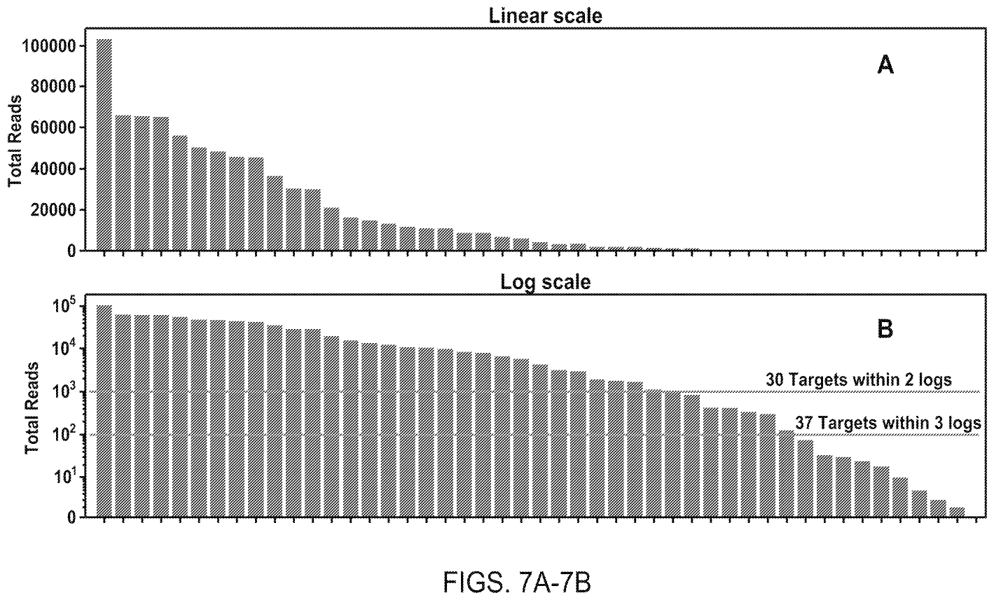

### DETAILED DESCRIPTION

### Definitions

The term “subject” refers to any individual who is the target of administration or treatment.

The subject can be a vertebrate, for example, a mammal Thus, the subject can be a human or veterinary patient. The term “patient” refers to a subject under the treatment of a clinician, e.g., physician. The subject can be either male or female.

The term “biological sample” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid. In preferred embodiments, the biological fluid is nipple aspirate fluid. The “biological sample” can comprise genomic DNA, or any other forms of nucleic acid.

The terms “peptide,” “protein,” and “polypeptide” are used interchangeably to refer to a natural or synthetic molecule comprising two or more amino acids linked by the carboxyl group of one amino acid to the alpha amino group of another.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203. In certain embodiments, useful MIP guide sequences hybridize to sequences that flank the nucleotide base or series of bases to be queried.

“Hybridization” refers to the process in which two single-stranded oligonucleotides bind non-covalently to form a stable double-stranded oligonucleotide. The term “hybridization” may also refer to triple-stranded hybridization. The resulting (usually) double-stranded oligonucleotide is a “hybrid” or “duplex.” “Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. In certain exemplary embodiments, hybridization takes place at room temperature.

“Amplifying” includes the production of copies of a nucleic acid molecule of the array or a nucleic acid molecule bound to a bead via repeated rounds of primed enzymatic synthesis.

“Nucleoside” as used herein includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g., described by Scheit, Nucleotide Analogs (John Wiley, New York, 1980); Uhlman and Peyman, Chemical Reviews, 90:543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structural Biology, 5:343-355 (1995); and the like. Exemplary types of polynucleotides that are capable of enhancing duplex stability include oligonucleotide phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides comprising probes or primers usually have lengths in the range of from 12 to 60 nucleotides, and more usually, from 18 to 40 nucleotides. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

“Sequencing” refers to determining the order of nucleotides (base sequences) in a nucleic acid sample, e.g. DNA or RNA. Many techniques are available such as Sanger sequencing and High Throughput Sequencing technologies (HTS). Sanger sequencing may involve sequencing via detection through (capillary) electrophoresis, in which up to 384 capillaries may be sequence analysed in one run. High throughput sequencing involves the parallel sequencing of thousands or millions or more sequences at once. HTS can be defined as Next Generation sequencing, i.e. techniques based on solid phase pyrosequencing or as Next-Next Generation sequencing (NGS) based on single nucleotide real time sequencing (SMRT).HTS technologies are available such as offered by Roche, Illumina and Applied Biosystems (Life Technologies). Further high throughput sequencing technologies are described by and/or available from Helicos, Pacific Biosciences, Complete Genomics, Ion Torrent Systems, Oxford Nanopore Technologies, Nabsys, ZS Genetics, GnuBio. Each of these sequencing technologies have their own way of preparing samples prior to the actual sequencing step. These steps may be included in the high throughput sequencing method. In certain cases, steps that are particular for the sequencing step may be integrated in the sample preparation protocol prior to the actual sequencing step for reasons of efficiency or economy. For instance, adapters that are ligated to fragments may contain sections that can be used in subsequent sequencing steps (so-called sequencing adapters). Or primers that are used to amplify a subset of fragments prior to sequencing may contain parts within their sequence that introduce sections that can later be used in the sequencing step, for instance by introducing through an amplification step a sequencing adapter or a capturing moiety in an amplicon that can be used in a subsequent sequencing step. Depending also on the sequencing technology used, amplification steps may be omitted.

“Multiplex sequencing” refers to a sequencing technique that allows for processing a large number of samples on a high-throughput instrument. For multiplex sequencing, individual “barcode” sequences are added to each sample so that nucleotide sequences from different samples can be distinguished by the unique barcode sequences embedded in each sample. With this technique, multiple DNA or RNA samples can be pooled, processed, sequenced, and analyzed simultaneously. “2D sequencing” or “1D2 sequencing” refers to a sequencing technology that enables reading both the sense and anti-sense strands (also known as template and complementary strands) in the single-molecule sequencing technologies, including the Nanopore Sequencing technology (Oxford Nanopore Technologies).

As used herein, a “dataset” is a set of data associated with a barcode or set of barcodes. Such data can include physical characteristics of a barcode or set of barcodes, such as primary sequence, homology to other sequences, melting temperature, GC content, propensity to form a hairpin, among other distinguishing characteristics or parameters. A dataset may be determined experimentally, calculated, or derived from information in other databases or publications.

As used herein, the term “alignment” refers to the identification of regions of similarity in a pair of sequences. For example, barcode sequences can be aligned, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), among others.

As used herein, a “sequencing read” refers to a sequence of nucleotides generated by sequencing a target nucleic acid.

By “cooperative nucleic acid” is meant a nucleic acid sequence which incorporates minimally a first nucleic acid sequence and a second nucleic acid sequence, wherein the second nucleic acid sequence hybridizes to the target nucleic acid downstream of the 3′ end of the first nucleic acid sequence. The 3′ end of the nucleic acid can be extendable, as discussed elsewhere herein. In one example, the first nucleic acid is a primer, and the second nucleic acid is a capture sequence. The first and second nucleic acid sequences can be separated by a linker, for example.

A “primer” is a nucleic acid that contains a sequence complementary to a region of a template nucleic acid strand and that primes the synthesis of a strand complementary to the template (or a portion thereof). Primers are typically, but need not be, relatively short, chemically synthesized oligonucleotides (typically, deoxyribonucleotides). In an amplification, e.g., a PCR amplification, a pair of primers typically define the 5′ ends of the two complementary strands of the nucleic acid target that is amplified. By “cooperative primer,” or first nucleic acid sequence, is meant a primer attached via a linker to a second nucleic acid sequence, also referred to as a capture sequence. The second nucleic acid sequence, or capture sequence, can hybridize to the template nucleic acid downstream of the 3′ end of the primer, or first nucleic acid sequence. By “normal primer” is meant a primer which does not have a capture sequence, or second nucleic acid sequence, attached to it via a linker.

By “target nucleic acid sequence,” which is also referred to herein as a “target nucleic acid region” is meant a sequence which hybridizes to the primer sequence, and is to be amplified and/or detected via sequencing.

“Downstream” is relative to the action of the polymerase during nucleic acid synthesis or extension. For example, when the Taq polymerase extends a primer, it adds bases to the 3′ end of the primer and will move towards a sequence that is “downstream from the 3′ end of the primer.”

The “Tm” (melting temperature) of a nucleic acid duplex under specified conditions is the temperature at which half of the nucleic acid sequences are disassociated and half are associated. As used herin, “isolated Tm” refers to the individual melting temperature of either the first or second nucleic acid sequence in the cooperative nucleic acid when not in the cooperative pair. “Effective Tm” refers to the resulting melting temperature of either the first or second nucleic acid when linked together.

The term “linker” means the composition joining the first and second nucleic acids to each other. The linker comprises at least one non-extendable moiety, but may also comprise extendable nucleic acids, and can be any length. The linker may be connected to the 3′ end, the 5′ end, or can be connected one or more bases from the end (“the middle”) of both the first and second nucleic acid sequences. The connection can be covalent, hydrogen bonding, ionic interactions, hydrophobic interactions, and the like. The term “non-extendable” has reference to the inability of the native Taq polymerase to recognize a moiety and thereby continue nucleic acid synthesis. A variety of natural and modified nucleic acid bases are recognized by the polymerase and are “extendable.” Examples of non-extendable moieties include among others, fluorophores, quenchers, polyethylene glycol, polypropylene glycol, polyethylene, polypropylene, polyamides, polyesters and others known to those skilled in the art. In some cases, even a nucleic acid base with reverse orientation (e.g. 5′ ACGT 3′ 3′A 5′ 5′ AAGT 3′) or otherwise rendered such that the Taq polymerase could not extend through it could be considered “non-extendable.”The term “non-nucleic acid linker” as used herein refers to a reactive chemical group that is capable of covalently attaching a first nucleic acid to a second nucleic acid, or more specifically, the primer to the capture sequence. Suitable flexible linkers are typically linear molecules in a chain of at least one or two atoms, more typically an organic polymer chain of 1 to 12 carbon atoms (and/or other backbone atoms) in length. Exemplary flexible linkers include polyethylene glycol, polypropylene glycol, polyethylene, polypropylene, polyamides, polyesters and the like.

### General

Disclosed herein is a method of preparing a target nucleic acid sequence for targeted amplicon sequencing comprising: a) providing at least one target nucleic acid sequence in a sample; b) exposing the target nucleic acid sequence to at least one pair of primer/adapter sequences, wherein each of the primer/adapter sequences comprise a region that hybridizes with the target nucleic acid sequence, as well as an adapter sequence that does not hybridize with the target nucleic acid sequence; c) amplifying the target nucleic acid in the presence of the primer/adapter sequence pair, thereby incorporating the adapter sequence into copies of the target nucleic acid sequence, creating a target nucleic acid/adapter sequence; d) purifying copies of the target nucleic acid/adapter sequence; and e) exposing the purified target nucleic acid/adapter sequence of step d) to reagents necessary for sequencing.

Generally speaking, the disclosed method relies on the incorporation of an adapter sequence into a copy of a target nucleic acid sequence which is to be sequenced. This is done by using a “primer/adapter sequence” which includes both the primer for amplification as well as the adapter for sequencing capture. These adapters are specific nucleic acid sequences that allow attachment of target nucleic acid sequences to the instrument sequencing substrate, such as a bead. Targets prepared with adapter sequences are referred to herein as a “target nucleic acid/adapter sequence,” and are used to create a library of targets.

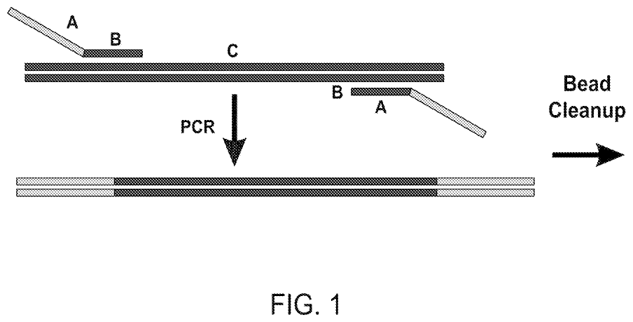

Adapter sequences needed for substrate attachment in NGS differ across instrument manufacturers. In FIG. 1, it can be seen that the adapter sequence (A) is included at the 5′ end of target specific primers (B), thereby forming a “primer/adapter sequence.” The adapters are then incorporated into the ends of the target nucleic acid sequences (C) with PCR, thereby forming a “target nucleic acid/adapter sequence.” Samples are then purified with beads or other methods to remove unincorporated primers and off-target amplification products. The adapters at the ends of the target sequences allow for the capture of the target nucleic acid sequence so that the target may be subsequently sequenced.

The methods disclosed herein are intended for NGS that relies on polymerase chain reaction (PCR) or other means of amplification to generate nucleic acid targets. This is often referred to as targeted amplicon sequencing and is used to sequence selected regions of a genome. This contrasts with shotgun sequencing methods that are intended to sequence all nucleic acid in a sample.

Traditional methods for targeted amplicon sequencing include multiple steps.

1) Amplicon generation. PCR amplifies selected targets. Primers for PCR may include partial sequencing adapter sequences.

2) Amplicon preparation. This may include cleaning and/or preparation with enzymes.

3) Adapter incorporation. This may be done via PCR or incubating with ligase or other enzymes.

4) Library cleaning. Unwanted products are removed using one or more methods such as magnetic beads or gel electrophoresis.

This process generally takes 7-8 hours with 2-4 hours of hands-on time. Additional steps may be necessary depending on the needs of the NGS method. The methods disclosed herein combines steps 1-3 above into a single step, reducing total preparation and user hands-on time. For example, the methods disclosed herein can reduce the total time required for library preparation prior to sequencing by 10, 15, 20, 25, 30, 35, 40, 45, 50, 55, 60, 75, 90, 105, 120, 150, 180, 240, or more minutes when compared with the previous methods of separately incorporating adapter sequences and amplifying the target nucleic acid prior to sequencing.

The technology is not limited to any particular sequencing platform, but is generally applicable and platform-independent. For example, the methods disclosed herein can be used with Illumina systems, as well as Life Technologies Ion Torrent and Qiagen GeneReader systems. In some embodiments, the technology is applicable to emulsion PCR-based methods, bead-based, and non-based methods, and thus finds use in the Life Technologies SOLiD systems and the Qiagen NGS sequencing platforms. Sequencers are discussed in more detail below.

As mentioned above, the methods disclosed herein are intended for NGS that relies on polymerase chain reaction (PCR) to generate nucleic acid targets. In some embodiments, target nucleic acid sequences (e.g., DNA or RNA) are isolated from a biological sample containing a variety of other components, such as proteins, lipids, and non-target nucleic acids. target nucleic acid sequences can be obtained from any material (e.g., cellular material (live or dead), extracellular material, viral material, environmental samples (e.g., metagenomic samples), synthetic material (e.g., amplicons such as provided by PCR or other amplification technologies)), obtained from an animal, plant, bacterium, archaeon, fungus, or any other organism. Biological samples for use in the present invention include viral particles or preparations thereof. target nucleic acid sequences can be obtained directly from an organism or from a biological sample obtained from an organism, e.g., from blood, urine, cerebrospinal fluid, seminal fluid, saliva, sputum, stool, hair, sweat, tears, skin, and tissue. Exemplary samples include, but are not limited to, whole blood, lymphatic fluid, serum, plasma, buccal cells, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.), washes (e.g., oral, nasopharyngeal, bronchial, bronchialalveolar, optic, rectal, intestinal, vaginal, epidermal, etc.), and/or other specimens.

Any tissue or body fluid specimen may be used as a source for nucleic acid for use in the technology, including forensic specimens, archived specimens, preserved specimens, and/or specimens stored for long periods of time, e.g., fresh-frozen, methanol/acetic acid fixed, or formalin-fixed paraffin embedded (FFPE) specimens and samples. Target nucleic acid sequences can also be isolated from cultured cells, such as a primary cell culture or a cell line. The cells or tissues from which target nucleic acid sequences are obtained can be infected with a virus or other intracellular pathogen. A sample can also be total RNA extracted from a biological specimen, a cDNA library, viral, or genomic DNA. A sample may also be isolated DNA from a non-cellular origin, e.g. amplified/isolated DNA that has been stored in a freezer.

Target nucleic acid sequences can be obtained, e.g., by extraction from a biological sample, e.g., by a variety of techniques such as those described by Maniatis, et al. (1982) Molecular Cloning: A Laboratory Manual, Cold Spring Harbor, N.Y. (see, e.g., pp. 280-281). In some embodiments, size selection of the nucleic acids is performed to remove very short fragments or very long fragments. Suitable methods select a size are known in the art.

The nucleic acid is amplified prior to sequencing. Any amplification method known in the art may be used, as long as it requires primers which can be used to incorporate the adapter sequence. Examples of amplification techniques that can be used include, but are not limited to, PCR, quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), hot start PCR, nested PCR, in situ polony PCR, in situ rolling circle amplification (RCA), bridge PCR, picotiter PCR, and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR), and nucleic acid based sequence amplification (NABSA). Other amplification methods that can be used herein include those described in U.S. Pat. Nos. 5,242,794; 5,494,810; 4,988,617; and 6,582,938.

Disclosed herein is a primer/adapter sequence pair, wherein the pair comprises both a forward primer and a reverse primer, both with the needed adapter sequence attached. One of skill in the art will understand how to design and validate primers. One of skill in the art will also be apprised of the other components needed to carry out PCR prior to sequencing.

The size of the primer/adapter sequence can be any size that supports the desired enzymatic manipulation of the primer, such as DNA amplification. A typical primer would be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primer/adapter sequence can be capable of forming a secondary structure. When the primer/adapter is capable of forming secondary structure, such as a hairpin, sequence elements can be located partially or completely outside the secondary structure, partially or completely inside the secondary structure, or in between sequences participating in the secondary structure. For example, when a primer/adapter sequence comprises a hairpin structure, sequence elements can be located partially or completely inside or outside the hybridizable sequences (the “stem”), including in the sequence between the hybridizable sequences (the “loop”). There can be different adapter sequences present in the same sample, and they can be attached to primer sequences which are identical to each other. Alternatively, the adapters can be identical to each other, which the primer sequences in the same sample differ from each other.

In some embodiments, the adapter sequences can contain a molecular binding site identification element to facilitate identification and isolation of the target nucleic acid sequence for downstream applications. Molecular binding as an affinity mechanism allows for the interaction between two molecules to result in a stable association complex. Molecules that can participate in molecular binding reactions include proteins, nucleic acids, carbohydrates, lipids, and small organic molecules such as ligands, peptides, or drugs.

When a nucleic acid molecular binding site is used as part of the adapter, it can be used to employ selective hybridization to isolate a target sequence. Selective hybridization may restrict substantial hybridization to target nucleic acids containing the adapter with the molecular binding site and capture nucleic acids, which are sufficiently complementary to the molecular binding site. Thus, through “selective hybridization” one can detect the presence of the target polynucleotide in an unpure sample containing a pool of many nucleic acids. An example of a nucleotide-nucleotide selective hybridization isolation system comprises a system with several capture nucleotides, which are complementary sequences to the molecular binding identification elements, and are optionally immobilized to a solid support.

The adapters can be used to immobilize the target nucleic acid to various solid supports, such as inside of a well of a plate, mono-dispersed spheres, beads, microarrays, or any other suitable support surface known in the art. The hybridized complementary adapter sequence attached on the solid support can be isolated by washing away the undesirable non-binding nucleic acids, leaving the desirable target sequences behind. If complementary adapter molecules are fixed to paramagnetic spheres or similar bead technology for isolation, then spheres can then be mixed in a tube together with the target polynucleotide containing the adapters (target nucleic acid/adapter sequence). When the adapter sequences have been hybridized with the complementary adapter sequences fixed to the spheres, undesirable molecules can be washed away while spheres are kept in the tube with a magnet or similar agent. The desired target molecules can be subsequently released by increasing the temperature, changing the pH, or by using any other suitable elution method known in the art.

In one embodiment, the primer/adapter sequence can be a cooperative primer, as disclosed in U.S. Pat. No. 10/093,966, herein incorporated by reference in its entirety for its teaching concerning cooperative primers. The cooperative primer can be modified so that an adapter sequence is incorporated on the 5′ end of the molecule. The cooperative primer can comprise a first nucleic acid sequence, wherein the first nucleic acid sequence is complementary to a first region of the target nucleic acid sequence, and wherein the first nucleic acid is extendable on the 3′ end; and a second nucleic acid sequence, wherein the second nucleic acid sequence is complementary to a second region of the target nucleic acid, such that in the presence of the target nucleic acid it hybridizes to the target nucleic acid downstream from the 3′ end of the first nucleic acid sequence; and a linker connecting said first and second nucleic acid sequences in a manner that allows both the said first and second nucleic acid sequences to hybridize to the target at the same time.

In some embodiments, the primer/adapter sequence can comprise other elements as well. For example, the primer/adapter can also comprise an index or barcode, as well as a universal sequencing primer. Indexes (also known as barcodes) are short sequences that allow individual samples to be identified after they are pooled together for the sequencing run. These are not necessary when only sequencing a single sample. Universal sequencing primers are universal for each target in the sequencing run and initiate sequencing by synthesis. This is not necessary for Ion Torrent which uses the reverse-complement of one of the adapters to initiate sequencing. These elements can be incorporated anywhere in the primer/adapter sequence, such as between the primer and adapter sequences, before, after, or within.

Regarding the index, or barcode, these can be used to associate a fragment with the template nucleic acid from which it was produced. In some embodiments, a unique index is a unique sequence of synthetic nucleotides or a unique sequence of natural nucleotides that allows for easy identification of the target nucleic acid within a complicated collection of oligonucleotides (e.g., fragments) containing various sequences. The indexes can be incorporated into the adapter sequences, such that they are within the adapter sequence. This ensures that homologous fragments can be detected based upon the unique indices that are attached to each fragment, thus further providing for unambiguous reconstruction of a consensus sequence. Homologous fragments may occur for example by chance due to genomic repeats, two fragments originating from homologous chromosomes, or fragments originating from overlapping locations on the same chromosome. Homologous fragments may also arise from closely related sequences (e.g., closely related gene family members, paralogs, orthologs, ohnologs, xenologs, and/or pseudogenes). Such fragments may be discarded to ensure that long fragment assembly can be computed unambiguously.

As used herein, the term “barcode” refers to a known nucleic acid sequence that allows some feature of a nucleic acid with which the barcode is associated to be identified. In some embodiments, the feature of the nucleic acid to be identified is the sample or source from which the nucleic acid is derived. In some embodiments, barcodes are at least 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, barcodes associated with some nucleic acids are of a different length than barcodes associated with other nucleic acids. In general, barcodes are of sufficient length and comprise sequences that are sufficiently different to allow the identification of samples based on barcodes with which they are associated. In some embodiments, a barcode and the sample source with which it is associated can be identified accurately after the mutation, insertion, or deletion of one or more nucleotides in the barcode sequence, such as the mutation, insertion, or deletion of 1, 2, 3, 4, 5, 6, 7, 8, 9, 10, or more nucleotides. In some embodiments, each barcode in a plurality of barcodes differs from every other barcode in the plurality at two or more nucleotide positions, such as at 2, 3, 4, 5, 6, 7, 8, 9, 10, or more positions. In some embodiments, the adapter sequence can include the barcode sequence. In some embodiments, methods of the technology further comprise identifying the sample or source from which a target nucleic acid is derived based on a barcode sequence to which the target nucleic acid is joined. In some embodiments, methods of the technology further comprise identifying the target nucleic acid based on a barcode sequence to which the target nucleic acid is joined. Some embodiments of the method further comprise identifying a source or sample of the target nucleotide sequence by determining a barcode nucleotide sequence. Some embodiments of the method further comprise molecular counting applications (e.g., digital barcode enumeration and/or binning) to determine expression levels or copy number status of desired targets. In general, a barcode may comprise a nucleic acid sequence that when joined to a target nucleic acid sequence serves as an identifier of the sample from which the target polynucleotide was derived.

In some embodiments, the primer/adapter sequence can also comprise a “universal” sequencing primer. A universal sequencing primer is a known sequence, e.g., for use as a primer binding site using a primer of a known sequence (e.g., complementary to the universal sequencing primer). While a target sequence of a primer, a barcode sequence of a primer, and/or a the sequence of the adapter might differ in embodiments of the technology, e.g., from fragment to fragment, from sample to sample, from source to source, or from region of interest to region of interest, embodiments of the technology provide that a universal sequencing primer is the same from fragment to fragment, from sample to sample, from source to source, or from region of interest to region of interest so that all fragments comprising the universal sequencing primer can be handled and/or treated in a same or similar manner, e.g., amplified, identified, sequenced, isolated, etc., using similar methods or techniques (e.g., using the same primer or probe).

In particular embodiments, the primer/adapter disclosed herein can comprise a universal sequencing primer (A), a barcode sequence (B), an adapter (C), and a target-specific sequence (D). While only C and D are required elements of the present invention, combinations of A, C, and D, or B, C, and D, or A, B, C, and D are all contemplated.

For example, if two regions of interest are to be sequenced (e.g., from the same or different sources or, e.g., from two different regions of the same nucleic acid, chromosome, gene, etc.), two primer/adapter pairs may be used, one primer pair comprising a first target-specific sequence for priming from the first region of interest and a first barcode to associate the first amplified product with the first region of interest, as well as an adapter for capture of the sequence; and a second primer pair comprising a second target-specific sequence for priming from the second region of interest and a second barcode to associate the second amplified product with the second region of interest, as well as an adapter for capture of the sequence. These two primer pairs, however, in some embodiments, will comprise the same universal sequencing primer for pooling and downstream processing together. Two or more universal sequencing primers may be used and, in general, the number of universal sequencing primers will be less than the number of target-specific sequences and/or barcode sequences for pooling of samples and treatment of pools as a single sample (batch).

Accordingly, in some embodiments, determining the first nucleotide subsequence and the second nucleotide subsequence comprises priming from a universal sequencing primer. In some embodiments determining the first nucleotide subsequence and the second nucleotide subsequence comprises terminating polymerization with a 3′-O-blocked nucleotide analog. For example, in some embodiments determining the first nucleotide subsequence and the second nucleotide subsequence comprises terminating polymerization with a 3′-O-alkynyl nucleotide analog, e.g., in some embodiments determining the first nucleotide subsequence and the second nucleotide subsequence comprises terminating polymerization with a 3′-O-propargyl nucleotide analog. In some embodiments determining the first nucleotide subsequence and the second nucleotide subsequence comprises terminating polymerization with a nucleotide analog comprising a reversible terminator.

The obtained short sequence reads are partitioned according to their barcode (e.g., de-multiplexed) and reads originating from the same samples, sources, regions of interest, etc. are binned together, e.g., saved to separate files or held in an organized data structure that allows binned reads to be identified as such. Then the binned short sequences are assembled into a consensus sequence. Sequence assembly can generally be divided into two broad categories: de novo assembly and reference genome mapping assembly. In de novo assembly, sequence reads are assembled together so that they form a new and previously unknown sequence. In reference genome mapping, sequence reads are assembled against an existing backbone sequence (e.g., a reference sequence, etc.) to build a sequence that is similar but not necessarily identical to the backbone sequence.

Thus, in some embodiments, target nucleic acid sequences corresponding to each region of interest are reconstructed using a de-novo assembly. To begin the reconstruction process, short reads are stitched together bioinformatically by finding overlaps and extending them to produce a consensus sequence. In some embodiments the method further comprises mapping the consensus sequence to a reference sequence. Methods of the technology take advantage of sequencing quality scores that represent base calling confidence to reconstruct full length fragments. In addition to de-novo assembly, fragments can be used to obtain phasing (assignment to homologous copies of chromosomes) of genomic variants by observing that consensus sequences originate from either one of the chromosomes.

There can be multiple primer/adapter pairs, so that multiplexing can occur. For example, there can be at least 2, 3, 4, 5, 6, 7, 8, 9, 10, 15, 20, 25, 30, 35, 40, 45, 50, 55, 60, 65, 70, 75, 80, 85, 90, 95, or 100 or more different primer/adapter sequence pairs present in the same sample.

The primer/adapters disclosed herein can hybridize in any way that is effective for amplification. The hybridization of two nucleic acids is affected by a number of conditions and parameters known to those of skill in the art. For example, the salt concentrations, pH, and temperature of the reaction all affect whether two nucleic acid molecules will hybridize. Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps.

For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000-fold excess. This type of assay can be performed at under conditions where both the limiting and non-limiting primer are for example, 10-fold or 100-fold or 1000-fold below their k_{d}, or where only one of the nucleic acid molecules is 10-fold or 100-fold or 1000-fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

After the NGS library is prepared, nucleic acid sequence data is generated (sequencing of the library takes place). Various embodiments of nucleic acid sequencing platforms (e.g., a nucleic acid sequencer) include components as described below. According to various embodiments, a sequencing instrument includes a fluidic delivery and control unit, a sample processing unit, a signal detection unit, and a data acquisition, analysis and control unit. Various embodiments of the instrument provide for automated sequencing that is used to gather sequence information from a plurality of sequences in parallel and/or substantially simultaneously.

In some embodiments, the fluidics delivery and control unit includes a reagent delivery system. The reagent delivery system includes a reagent reservoir for the storage of various reagents. The reagents can include RNA-based primers, forward/reverse DNA primers, nucleotide mixtures (e.g., compositions comprising nucleotide analogs as provided herein) for sequencing-by-synthesis, buffers, wash reagents, blocking reagents, stripping reagents, and the like. Additionally, the reagent delivery system can include a pipetting system or a continuous flow system that connects the sample processing unit with the reagent reservoir.

In some embodiments, the sample processing unit includes a sample chamber, such as flow cell, a substrate, a micro-array, a multi-well tray, or the like. The sample processing unit can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber. In some embodiments, the signal detection unit can include an imaging or detection sensor. For example, the imaging or detection sensor (e.g., a fluorescence detector or an electrical detector) can include a CCD, a CMOS, an ion sensor, such as an ion sensitive layer overlying a CMOS, a current detector, or the like. The signal detection unit can include an excitation system to cause a probe, such as a fluorescent dye, to emit a signal. The detection system can include an illumination source, such as arc lamp, a laser, a light emitting diode (LED), or the like. In particular embodiments, the signal detection unit includes optics for the transmission of light from an illumination source to the sample or from the sample to the imaging or detection sensor. Alternatively, the signal detection unit may not include an illumination source, such as for example, when a signal is produced spontaneously as a result of a sequencing reaction. For example, a signal can be produced by the interaction of a released moiety, such as a released ion interacting with an ion sensitive layer, or a pyrophosphate reacting with an enzyme or other catalyst to produce a chemiluminescent signal. In another example, changes in an electrical current, voltage, or resistance are detected without the need for an illumination source.

In some embodiments, a data acquisition analysis and control unit monitors various system parameters. The system parameters can include temperature of various portions of the instrument, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of the instruments and systems are used to practice sequencing methods such as sequencing by synthesis, single molecule methods, and other sequencing techniques. Sequencing by synthesis can include the incorporation of dye labeled nucleotides, chain termination, ion/proton sequencing, pyrophosphate sequencing, or the like. Single molecule techniques can include staggered sequencing, where the sequencing reactions is paused to determine the identity of the incorporated nucleotide.

Particular sequencing technologies contemplated by the technology are next-generation sequencing (NGS) methods that share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems.

Also contemplated herein is pyrosequencing. In pyrosequencing (Voelkerding et al., Clinical Chem., 55: 641-658, 2009; MacLean et al., Nature Rev. Microbiol., 7: 287-296; U.S. Pat. Nos. 6,210,891; 6,258,568; each herein incorporated by reference in its entirety), the NGS fragment library is clonally amplified in-situ by capturing single template molecules with beads bearing oligonucleotides complementary to the adapter sequences. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 106 sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each herein incorporated by reference in its entirety), sequencing data are produced in the form of shorter-length reads. In this method, the fragments of the NGS fragment library are captured on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 100 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. Nos. 5,912,148; 6,130,073; each herein incorporated by reference in their entirety) also involves clonal amplification of the NGS fragment library by emulsion PCR. This can be done using the primer/adapter sequences disclosed herein. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, HeliScope by Helicos BioSciences is employed (Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each herein incorporated by reference in their entirety). Sequencing is achieved by addition of polymerase and serial addition of fluorescently-labeled dNTP reagents. Incorporation events result in a fluor signal corresponding to the dNTP, and signal is captured by a CCD camera before each round of dNTP addition. Sequence read length ranges from 25-50 nucleotides, with overall output exceeding 1 billion nucleotide pairs per analytical run.

In some embodiments, 454 sequencing by Roche is used (Margulies et al. (2005) Nature 437: 376-380). 454 sequencing involves two steps. In the first step, DNA is sheared into fragments of approximately 300-800 base pairs and the fragments are blunt ended. The primer/adapter sequences disclosed herein can be used with this method. The fragments can be attached to DNA capture beads, e.g., streptavidin-coated beads using, e.g., an adapter that contains a 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a fragment of the NGS fragment library to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used.

Another exemplary nucleic acid sequencing approach that may be adapted for use with the present invention was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “HIGH THROUGHPUT NUCLEIC ACID SEQUENCING BY EXPANSION,” filed Jun. 19, 2008, which is incorporated herein in its entirety.

Other single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which fragments of the NGS fragment library are immobilized, primed, then subjected to strand extension using a fluorescently-modified polymerase and florescent acceptor molecules, resulting in detectible fluorescence resonance energy transfer (FRET) upon nucleotide addition.

Another real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. Nos. 7,170,050; 7,302,146; 7,313,308; 7,476,503; all of which are herein incorporated by reference) utilizes reaction wells 50-100 nm in diameter and encompassing a reaction volume of approximately 20 zeptoliters (10-211). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10-211). At this volume, the activity of a single molecule can be detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background.

In some embodiments, nanopore sequencing is used (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

In some embodiments, a sequencing technique uses a chemical-sensitive field effect transistor (chemFET) array to sequence DNA (for example, as described in US Patent Application Publication No. 20090026082). In one example of the technique, DNA molecules are placed into reaction chambers, and the template molecules are hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Processes and systems for such real time sequencing that may be adapted for use with the invention are described in, for example, U.S. Pat. No. 7,405,281, entitled “Fluorescent nucleotide analogs and uses therefor”, issued Jul. 29, 2008 to Xu et al.; U.S. Pat. No. 7,315,019, entitled “Arrays of optical confinements and uses thereof”, issued Jan. 1, 2008 to Turner et al.; U.S. Pat. No. 7,313,308, entitled “Optical analysis of molecules”, issued Dec. 25, 2007 to Turner et al.; U.S. Pat. No. 7,302,146, entitled “Apparatus and method for analysis of molecules”, issued Nov. 27, 2007 to Turner et al.; and U.S. Pat. No. 7,170,050, entitled “Apparatus and methods for optical analysis of molecules”, issued Jan. 30, 2007 to Turner et al.; and U.S. Pat. Pub. Nos. 20080212960, entitled “Methods and systems for simultaneous real-time monitoring of optical signals from multiple sources”, filed Oct. 26, 2007 by Lundquist et al.; 20080206764, entitled “Flowcell system for single molecule detection”, filed Oct. 26, 2007 by Williams et al.; 20080199932, entitled “Active surface coupled polymerases”, filed Oct. 26, 2007 by Hanzel et al.; 20080199874, entitled “CONTROLLABLE STRAND SCISSION OF MINI CIRCLE DNA”, filed Feb. 11, 2008 by Otto et al.; 20080176769, entitled “Articles having localized molecules disposed thereon and methods of producing same”, filed Oct. 26, 2007 by Rank et al.; 20080176316, entitled “Mitigation of photodamage in analytical reactions”, filed Oct. 31, 2007 by Eid et al.; 20080176241, entitled “Mitigation of photodamage in analytical reactions”, filed Oct. 31, 2007 by Eid et al.; 20080165346, entitled “Methods and systems for simultaneous real-time monitoring of optical signals from multiple sources”, filed Oct. 26, 2007 by Lundquist et al.; 20080160531, entitled “Uniform surfaces for hybrid material substrates and methods for making and using same”, filed Oct. 31, 2007 by Korlach; 20080157005, entitled “Methods and systems for simultaneous real-time monitoring of optical signals from multiple sources”, filed Oct. 26, 2007 by Lundquist et al.; 20080153100, entitled “Articles having localized molecules disposed thereon and methods of producing same”, filed Oct. 31, 2007 by Rank et al.; 20080153095, entitled “CHARGE SWITCH NUCLEOTIDES”, filed Oct. 26, 2007 by Williams et al.; 20080152281, entitled “Substrates, systems and methods for analyzing materials”, filed Oct. 31, 2007 by Lundquist et al.; 20080152280, entitled “Substrates, systems and methods for analyzing materials”, filed Oct. 31, 2007 by Lundquist et al.; 20080145278, entitled “Uniform surfaces for hybrid material substrates and methods for making and using same”, filed Oct. 31, 2007 by Korlach; 20080128627, entitled “SUBSTRATES, SYSTEMS AND METHODS FOR ANALYZING MATERIALS”, filed Aug. 31, 2007 by Lundquist et al.; 20080108082, entitled “Polymerase enzymes and reagents for enhanced nucleic acid sequencing”, filed Oct. 22, 2007 by Rank et al.; 20080095488, entitled “SUBSTRATES FOR PERFORMING ANALYTICAL REACTIONS”, filed Jun. 11, 2007 by Foquet et al.; 20080080059, entitled “MODULAR OPTICAL COMPONENTS AND SYSTEMS INCORPORATING SAME”, filed Sep. 27, 2007 by Dixon et al.; 20080050747, entitled “Articles having localized molecules disposed thereon and methods of producing and using same”, filed Aug. 14, 2007 by Korlach et al.; 20080032301, entitled “Articles having localized molecules disposed thereon and methods of producing same”, filed Mar. 29, 2007 by Rank et al.; 20080030628, entitled “Methods and systems for simultaneous real-time monitoring of optical signals from multiple sources”, filed Feb. 9, 2007 by Lundquist et al.; 20080009007, entitled “CONTROLLED INITIATION OF PRIMER EXTENSION”, filed Jun. 15, 2007 by Lyle et al.; 20070238679, entitled “Articles having localized molecules disposed thereon and methods of producing same”, filed Mar. 30, 2006 by Rank et al.; 20070231804, entitled “Methods, systems and compositions for monitoring enzyme activity and applications thereof”, filed Mar. 31, 2006 by Korlach et al.; 20070206187, entitled “Methods and systems for simultaneous real-time monitoring of optical signals from multiple sources”, filed Feb. 9, 2007 by Lundquist et al.; 20070196846, entitled “Polymerases for nucleotide analog incorporation”, filed Dec. 21, 2006 by Hanzel et al.; 20070188750, entitled “Methods and systems for simultaneous real-time monitoring of optical signals from multiple sources”, filed Jul. 7, 2006 by Lundquist et al.; 20070161017, entitled “MITIGATION OF PHOTODAMAGE IN ANALYTICAL REACTIONS”, filed Dec. 1, 2006 by Eid et al.; 20070141598, entitled “Nucleotide Compositions and Uses Thereof”, filed Nov. 3, 2006 by Turner et al.; 20070134128, entitled “Uniform surfaces for hybrid material substrate and methods for making and using same”, filed Nov. 27, 2006 by Korlach; 20070128133, entitled “Mitigation of photodamage in analytical reactions”, filed Dec. 2, 2005 by Eid et al.; 20070077564, entitled “Reactive surfaces, substrates and methods of producing same”, filed Sep. 30, 2005 by Roitman et al.; 20070072196, entitled “Fluorescent nucleotide analogs and uses therefore”, filed Sep. 29, 2005 by Xu et al; and 20070036511, entitled “Methods and systems for monitoring multiple optical signals from a single source”, filed Aug. 11, 2005 by Lundquist et al.; and Korlach et al. (2008) “Selective aluminum passivation for targeted immobilization of single DNA polymerase molecules in zero-mode waveguide nanostructures” PNAS 105(4): 1176-81, all of which are herein incorporated by reference in their entireties.

In some embodiments, a computer-based analysis program is used to translate the raw data generated by the detection assay (e.g., sequencing reads) into data of predictive value for an end user (e.g., medical personnel). The user can access the predictive data using any suitable means. Thus, in some preferred embodiments, the present technology provides the further benefit that the user, who is not likely to be trained in genetics or molecular biology, need not understand the raw data. The data is presented directly to the end user in its most useful form. The user is then able to immediately utilize the information to determine useful information (e.g., in medical diagnostics, research, or screening).

Some embodiments provide a system for reconstructing a nucleic acid sequence. The system can include a nucleic acid sequencer, a sample sequence data storage, a reference sequence data storage, and an analytics computing device/server/node. In some embodiments, the analytics computing device/server/node can be a workstation, mainframe computer, personal computer, mobile device, etc. The nucleic acid sequencer can be configured to analyze (e.g., interrogate) a nucleic acid fragment (e.g., single fragment, mate-pair fragment, paired-end fragment, etc.) utilizing all available varieties of techniques, platforms or technologies to obtain nucleic acid sequence information, in particular the methods as described herein using compositions provided herein. In some embodiments, the nucleic acid sequencer is in communications with the sample sequence data storage either directly via a data cable (e.g., serial cable, direct cable connection, etc.) or bus linkage or, alternatively, through a network connection (e.g., Internet, LAN, WAN, VPN, etc.). In some embodiments, the network connection can be a “hardwired” physical connection. For example, the nucleic acid sequencer can be communicatively connected (via Category 5 (CAT5), fiber optic or equivalent cabling) to a data server that is communicatively connected (via CAT5, fiber optic, or equivalent cabling) through the Internet and to the sample sequence data storage. In some embodiments, the network connection is a wireless network connection (e.g., Wi-Fi, WLAN, etc.), for example, utilizing an 802.11 a/b/g/n or equivalent transmission format. In practice, the network connection utilized is dependent upon the particular requirements of the system. In some embodiments, the sample sequence data storage is an integrated part of the nucleic acid sequencer.

In some embodiments, the sample sequence data storage is any database storage device, system, or implementation (e.g., data storage partition, etc.) that is configured to organize and store nucleic acid sequence read data generated by nucleic acid sequencer such that the data can be searched and retrieved manually (e.g., by a database administrator or client operator) or automatically by way of a computer program, application, or software script. In some embodiments, the reference data storage can be any database device, storage system, or implementation (e.g., data storage partition, etc.) that is configured to organize and store reference sequences (e.g., whole or partial genome, whole or partial exome, SNP, gen, etc.) such that the data can be searched and retrieved manually (e.g., by a database administrator or client operator) or automatically by way of a computer program, application, and/or software script. In some embodiments, the sample nucleic acid sequencing read data can be stored on the sample sequence data storage and/or the reference data storage in a variety of different data file types/formats, including, but not limited to: *.txt, *.fasta, *.csfasta, *seq.txt, *qseq.txt, *.fastq, *.sff, *prb.txt, *.sms, *srs and/or *.qv.

In some embodiments, the sample sequence data storage and the reference data storage are independent standalone devices/systems or implemented on different devices. In some embodiments, the sample sequence data storage and the reference data storage are implemented on the same device/system. In some embodiments, the sample sequence data storage and/or the reference data storage can be implemented on the analytics computing device/server/node. The analytics computing device/server/node can be in communications with the sample sequence data storage and the reference data storage either directly via a data cable (e.g., serial cable, direct cable connection, etc.) or bus linkage or, alternatively, through a network connection (e.g., Internet, LAN, WAN, VPN, etc.). In some embodiments, analytics computing device/server/node can host a reference mapping engine, a de novo mapping module, and/or a tertiary analysis engine. In some embodiments, the reference mapping engine can be configured to obtain sample nucleic acid sequence reads from the sample data storage and map them against one or more reference sequences obtained from the reference data storage to assemble the reads into a sequence that is similar but not necessarily identical to the reference sequence using all varieties of reference mapping/alignment techniques and methods. The reassembled sequence can then be further analyzed by one or more optional tertiary analysis engines to identify differences in the genetic makeup (genotype), gene expression or epigenetic status of individuals that can result in large differences in physical characteristics (phenotype). For example, in some embodiments, the tertiary analysis engine can be configured to identify various genomic variants (in the assembled sequence) due to mutations, recombination/crossover or genetic drift. Examples of types of genomic variants include, but are not limited to: single nucleotide polymorphisms (SNPs), copy number variations (CNVs), insertions/deletions (Indels), inversions, etc. The optional de novo mapping module can be configured to assemble sample nucleic acid sequence reads from the sample data storage into new and previously unknown sequences. It should be understood, however, that the various engines and modules hosted on the analytics computing device/server/node can be combined or collapsed into a single engine or module, depending on the requirements of the particular application or system architecture. Moreover, in some embodiments, the analytics computing device/server/node can host additional engines or modules as needed by the particular application or system architecture.

In some embodiments, the mapping and/or tertiary analysis engines are configured to process the nucleic acid and/or reference sequence reads in color space. In some embodiments, the mapping and/or tertiary analysis engines are configured to process the nucleic acid and/or reference sequence reads in base space. It should be understood, however, that the mapping and/or tertiary analysis engines disclosed herein can process or analyze nucleic acid sequence data in any schema or format as long as the schema or format can convey the base identity and position of the nucleic acid sequence.

In some embodiments, the sample nucleic acid sequencing read and referenced sequence data can be supplied to the analytics computing device/server/node in a variety of different input data file types/formats, including, but not limited to: *.txt, *.fasta, *.csfasta, *seq.txt, *qseq.txt, *.fastq, *.sff, *prb.txt, *.sms, *srs and/or *.qv.

Furthermore, a client terminal can be a thin client or thick client computing device. In some embodiments, client terminal can have a web browser that can be used to control the operation of the reference mapping engine, the de novo mapping module and/or the tertiary analysis engine. That is, the client terminal can access the reference mapping engine, the de novo mapping module and/or the tertiary analysis engine using a browser to control their function. For example, the client terminal can be used to configure the operating parameters (e.g., mismatch constraint, quality value thresholds, etc.) of the various engines, depending on the requirements of the particular application. Similarly, client terminal can also display the results of the analysis performed by the reference mapping engine, the de novo mapping module and/or the tertiary analysis engine.

The present technology also encompasses any method capable of receiving, processing, and transmitting the information to and from laboratories conducting the assays, information provides, medical personal, and subjects.

The technology is not limited to particular uses, but finds use in a wide range of research (basic and applied), clinical, medical, and other biological, biochemical, and molecular biological applications. Some exemplary uses of the technology include genetics, genomics, and/or genotyping, e.g., of plants, animals, and other organisms, e.g., to identify haplotypes, phasing, and/or linkage of mutations and/or alleles. Particular and non-limiting illustrative examples in the human medical context include testing for cystic fibrosis and fragile X syndrome.

In addition, the technology finds use in the field of infectious disease, e.g., in identifying infectious agents such as viruses, bacteria, fungi, etc., and in determining viral types, families, species, and/or quasi-species, and to identify haplotypes, phasing, and/or linkage of mutations and/or alleles. A particular and non-limiting illustrative example in the area of infectious disease is characterization of human immunodeficiency virus (HIV) genetic elements and identifying haplotypes, phasing, and/or linkage of mutations and/or alleles. Other particular and non-limiting illustrative examples in the area of infectious disease include characterizing antibiotic resistance determinants; tracking infectious organisms for epidemiology; monitoring the emergence and evolution of resistance mechanisms; identifying species, sub-species, strains, extra-chromosomal elements, types, etc. associated with virulence, monitoring the progress of treatments, etc.

In some embodiments, the technology finds use in transplant medicine, e.g., for typing of the major histocompatibility complex (MHC), typing of the human leukocyte antigen (HLA), and for identifying haplotypes, phasing, and/or linkage of mutations and/or alleles associated with transplant medicine (e.g., to identify compatible donors for a particular host needing a transplant, to predict the chance of rejection, to monitor rejection, to archive transplant material, for medical informatics databases, etc.).

In some embodiments, the technology finds use in oncology and fields related to oncology. Particular and non-limiting illustrative examples in the area of oncology are identifying genetic and/or genomic aberrations related to cancer, predisposition to cancer, and/or treatment of cancer. For example, in some embodiments the technology finds use in detecting the presence of a chromosomal translocation associated with cancer; and in some embodiments the technology finds use in identifying novel gene fusion partners to provide cancer diagnostic tests. In some embodiments, the technology finds use in cancer screening, cancer diagnosis, cancer prognosis, measuring minimal residual disease, and selecting and/or monitoring a course of treatment for a cancer.

In some embodiments, the technology finds use in characterizing nucleotide sequences. For example, in some embodiments, the technology finds use in detecting insertions and/or deletions (“indels”) in a nucleotide (e.g., genome, gene, etc.) sequence. It is contemplated that the technology described herein provides improved indel detection relative to conventional technologies. In addition, the technology finds use in detecting short tandem repeats (STRs), inversions, large insertions, and in sequencing repetitive (e.g., highly repetitive) regions of a nucleotide sequence (e.g., of a genome).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Next Generation Sequencing Library Preparation Using Primer/Adapter Complexes

Methods

Oligonucleotides

Oligonucleotides consisted of up to four segments of the form [Platform-specific adapter][index or barcode][sequencing primer][target-specific primer]. Platform-specific adapters allow attachment of the sequencing target to the sequencing platform substrate. Indexes (also known as barcodes) are short sequences that allow individual samples to be identified after they are pooled together for the sequencing run. These are not necessary when only sequencing a single sample and were omitted for the Ion Torrent experiment. Sequencing primers are universal for each target in the sequencing run and initiate sequencing by synthesis. This is not necessary for Ion Torrent which uses the reverse-complement of one of the adapters to initiate sequencing. The target-specific primers allow amplification of the targets by PCR. Table 1 provides the sequences that were used.

Oligonucleotides were ordered from either LGC Biosearch Technologies or Integrated DNA Technologies, Inc. with salt-free purification.

PCR Amplification

PCR reactions consisted of 20-25 ng purified DNA (extracted from Zea Mays, Mo17 line), 10 μl 2× BHQ Probe Master Mix NO ROX (LGC, Catalog #: KBS-1040-006), 10 nM each primer, and 0.5 μM EvaGreen® Dye, 20× (Biotium, Catalog #: 31000) in a 20 μl reaction volume. Multiple identical reactions were pooled together (4-10) before performing bead cleaning. 100 targets (200 primers) were included in the library for the Ion Torrent platform and 50 targets (100 primers) for the Illumina platform.

The reaction protocol consisted on 15 min hot-start polymerase deactivation at 95° C. followed by 45 cycles of 5 s at 95° C. and 3 m at 55° C. (Illumina platform) or 5 s at 95° C. and 2 m at 62° C. (Ion Torrent platform). PCR was performed with Mic qPCR thermal cyclers (Bio Molecular Systems). Total PCR time was about 3 hours or less.

The PCR Protocol for Ion Torrent is shown in Table 1:

TABLE 1

| Stage | Temp ° C. | Time | | Cycles |
|---|---|---|---|---|
| Activation | 95 | 15 | m | 1 |
| PCR | 95 | 5 | s | 45 |
| | 62 | 2 | m | |

| | |
|---|---|
| Total Time | 2 h 22 m |

Table 2 shows PCR protocol for Illumina. The Illumina platform adapters are longer, requiring longer annealing times for PCR.

TABLE 2

| Stage | Temp ° C. | Time | | Cycles |
|---|---|---|---|---|
| Activation | 95 | 15 | m | 1 |
| PCR | 95 | 5 | s | 45 |
| | 62 | 3 | m | |

| | |
|---|---|
| Total Time | 3 h 14 m |

Bead Purification

Beads were purified with sbeadex™ particles suspension SAB and eluted with Elution buffer SAB (LGC). Bead suspension was added to the sample at a ratio of 1.0, mixing by pipetting 10 times. Sample was incubated with the beads for 5 m. Bead mixture was transferred to a magnetic tube rack and beads were allowed to migrate to the magnet for 2 minutes. Remaining solution was discarded and beads were washed twice with 70% ethanol, incubating for 30 seconds each. Sample tubes were removed from the magnetic tube rack and 40 to 60 ul of elution buffer was mixed with the beads by pipetting 10 times. Samples were incubated with the elution buffer for at least 2 minutes. Sample tubes were returned to the magnetic rack and beads allowed to migrate for 1 minute. Elution buffer containing the desired PCR products were then removed with a pipette and transferred to a new tube. In the case of the Illumina platform, this procedure was repeated on additional time.

Quantification and Purification Verification

Samples were quantified with a Qubit fluorometer using Qubit lx dsDNA HS Assay Kit (ThermoFisher Scientific). Library purity was verified with a Fragment Analyzer (Agilent).

Sequencing

Sequencing was performed with either an Ion Proton™ System (ThermoFisher Scientific) or a MiSeq (Illumina). 25% PhiX Control v2 (Illumina, Catalog #: FC-110-3001) was spiked into the reaction for the Illumina system and the Reagent Kit v2 Nano (Illumina, Catalog #: MS-102-2002) was used with 2× 150 bp read chemistry.

Table 3 shows target coverage summary This table summarizes the coverage distribution for the Ion Torrent library:

TABLE 3

| | % Targets | # Reads |
|---|---|---|
| 1.0X Mean | 40 | 6608 |
| 0.5X Mean | 57 | 3304 |
| 0.2X Mean | 69 | 1322 |

Table 4 shows variants called at the SNP of interest for the Ion Torrent platform. SNP names have been deidentified.

TABLE 4

| SNP | Ref | Alt |
|---|---|---|
| SNP47 | T | C |
| SNP33 | C | G |
| SNP6 | A | G |
| SNP54 | C | A |
| SNP31 | A | G |
| SNP55 | G | A |
| SNP58 | C | T |
| SNP28 | T | C |
| SNP9 | G | A |
| SNP20 | C | T |
| SNP13 | A | G |
| SNP66 | T | C |
| SNP8 | C | A |
| SNP21 | C | T |
| SNP42 | A | G |
| SNP12 | G | A |
| SNP40 | T | C |
| SNP57 | G | A |
| SNP24 | C | G |
| SNP3 | T | G |
| SNP44 | C | T |
| SNP17 | C | G |
| SNP14 | C | T |
| SNP50 | C | T |
| SNP33 | C | T |
| SNP29 | T | C |
| SNP2 | G | A |
| SNP61 | A | T |
| SNP56 | G | A |
| SNP49 | A | G |
| SNP22 | C | A |
| SNP39 | G | A |
| SNP55 | C | T |
| SNP5 | T | C |
| SNP38 | C | T |
| SNP57 | A | G |
| SNP39 | G | A |
| SNP54 | C | G |
| SNP41 | A | G |
| SNP36 | A | G |
| SNP53 | A | C |
| SNP67 | A | G |
| SNP64 | T | C |
| SNP36 | A | T |
| SNP23 | T | C |
| SNP43 | C | T |
| SNP1 | T | C |
| SNP7 | G | A |
| SNP32 | T | A |
| SNP60 | G | C |
| SNP48 | C | T |
| SNP70 | C | A |
| SNP27 | A | G |
| SNP30 | G | A |
| SNP10 | C | A |
| SNP69 | A | C |
| SNP4 | A | G |
| SNP51 | A | G |
| SNP63 | C | T |
| SNP52 | G | A |
| SNP25 | A | G |
| SNP62 | A | G |
| SNP15 | C | T |
| SNP59 | C | T |
| SNP26 | T | C |
| SNP18 | C | T |
| SNP65 | C | G |
| SNP68 | C | T |
| SNP46 | C | T |
| SNP11 | C | T |

Variants called at the SNP of interest for the Ion Torrent platform. SNP names have been deidentified.

Data Analysis

Data from the Ion Torrent platform was analyzed with a combination of Ion Torrent tools (Torrent Suite) and open source bioinformatics tools. Illumina data was analyzed entirely with open source bioinformatics tools. Open source tools include samtools and bcftools, fastp, and bwa.

1. A method of preparing a target nucleic acid sequence for targeted amplicon sequencing comprising:
a. providing at least one target nucleic acid sequence in a sample;
b. exposing the target nucleic acid sequence to at least one pair of primer/adapter sequences, wherein each of the primer/adapter sequences comprise a region that hybridizes with the target nucleic acid sequence, as well as an adapter sequence that does not hybridize with the target nucleic acid sequence;
c. amplifying the target nucleic acid in the presence of the primer/adapter sequence pair, thereby incorporating the adapter sequence into copies of the target nucleic acid sequence, creating a target nucleic acid/adapter sequence;
d. purifying copies of the target nucleic acid/adapter sequence; and
e. exposing the purified target nucleic acid/adapter sequence of step d) to reagents necessary for sequencing.

2. The method of claim 1, wherein the primer/adapter sequence pair comprises one forward primer and one reverse primer.

3. The method of claim 1, wherein said sequencing comprises using a Next Generation Sequencer (NGS).

4. The method of claim 3, wherein the NGS comprises Illumina sequencing, Roche 454 sequencing, Ion Torrent sequencing, or SOLiD sequencing.

5. The method of claim 1, wherein the adapter sequence portion of the primer/adapter sequence is between 5-30 nucleotide bases in length.

6. The method of claim 1, wherein the adapter portion of the primer/adapter sequence is 5′ of the primer sequence.

7. The method of claim 1, wherein there are multiple target nucleic acid sequences in the sample.

8. The method of claim 7, wherein the target nucleic acid sequences are exposed to more than one primer/adapter sequence pairs.

9. The method of claim 8, wherein the primer/adapter sequence pairs differ from each other in the region that hybridizes with the target nucleic acid sequence, but the adapter sequences are identical.

10. The method of claim 8, wherein the primer/adapter sequence pairs differ from each other in the adapter sequence, but the regions that hybridizes with the target nucleic acid sequence are identical.

11. The method of claim 8, wherein the primer/adapter sequence pairs differ from each other in the region that hybridizes with the target nucleic acid sequence, and the adapter sequences are also different.

12. The method of claim 8 wherein there are at least 50 different primer/adapter sequence pairs present.

13. The method of claim 12, wherein there are at least 100 different primer/adapter sequence pairs present.

14. The method of claim 1, wherein the sample comprises non-target nucleic acid sequences.

15. The method of claim 7, wherein the target nucleic acid sequences are different from each other.

16. The method of claim 1, wherein the sample comprises genomic DNA.

17. The method of claim 1, wherein the primer portion of the primer/adapter sequence is a cooperative nucleic acid molecule comprising:
a. a first nucleic acid sequence, wherein the first nucleic acid sequence is complementary to a first region of a target nucleic acid, and wherein the first nucleic acid is extendable on the 3′ end;
b. a second nucleic acid sequence, wherein the second nucleic acid sequence is complementary to a second region of the target nucleic acid, such that in the presence of the target nucleic acid it hybridizes to the target nucleic acid downstream from the 3′ end of the first nucleic acid sequence;
c. a linker connecting said first and second nucleic acid sequences in a manner that allows both the said first and second nucleic acid sequences to hybridize to the target at the same time.

18. The method of claim 1, wherein, the primer/adapter sequence, in addition to comprising a region that hybridizes with the target nucleic acid sequence and an adapter sequence that does not hybridize with the target nucleic acid sequence, further comprises a barcode region.

19. The method of claim 1, wherein, the primer/adapter sequence, in addition to comprising a region that hybridizes with the target nucleic acid sequence and an adapter sequence that does not hybridize with the target nucleic acid sequence, further comprises a sequencing primer region.

20. The method of claim 1, wherein, the primer/adapter sequence, in addition to comprising a region that hybridizes with the target nucleic acid sequence and an adapter sequence that does not hybridize with the target nucleic acid sequence, further comprises a sequencing primer region and a barcode region.

21. The method of claim 19, wherein the sequencing primer region of the primer/adapter sequence remains the same in different primer/adapter sequences exposed to the same sample, but the region that hybridizes with the target nucleic acid sequence is different for different target nucleic acid sequences in the sample.

22. The method of claim 18, wherein different primer/adapter sequences comprise the same barcode in a single sample.

23. The method of claim 1, wherein purification of the target nucleic acid/adapter sequence takes place by using beads.
